(19) United States
(12) Patent Application Publication
(10) Pub. No.: US 2023/0395254 A1
(43) Pub. Date: Dec. 7, 2023

(54) Nausea and Vomiting Management System

(71) Applicant: Takeda Pharmaceutical Company Limited, Osaka-shi, Osaka (JP)

(72) Inventors: Jordan Brayanov, Cambridge, MA (US); Cristina Menchero Almansa, Cambridge, MA (US); Meena Subramanyam, Cambridge, MA (US)

(73) Assignee: Takeda Pharmaceutical Company Limited, Osaka-shi, Osaka (JP)

(21) Appl. No.: 18/006,967

(22) PCT Filed: Jul. 27, 2021
(86) PCT No.: PCT/JP2021/027677

§ 371 Date: Jan. 26, 2023

Related U.S. Application Data
(60) Provisional application No. 63/057,038, filed on Jul. 27, 2020.

(51) Int. Cl. G16H 50/20; G16H 20/17; A61B 5/0205; A61B 5/00; G16H 40/67

(52) U.S. Cl. CPC G16H 50/20; G16H 20/17; A61B 5/02055; A61B 5/7475; G16H 40/67

(57) ABSTRACT

A method (500) for managing nausea includes receiving, from one or more monitoring devices (200), patient parameter data (212) of a patient (10). The patient parameter data is collected by the one or more monitoring devices. The method also includes determining, based on the patient parameter data of the patient, whether the patient is experiencing or about to experience an episode of nausea. When the patient is experiencing or about to experience an episode of nausea, the method also includes communicating one or more therapy signals (302, 304, 306, 308) to a receiver device (12, 21, 400) in communication with the computing device.

### TECHNICAL FIELD

This disclosure relates to managing nausea and/or vomiting.

### BACKGROUND

Nausea is feeling an urge to vomit. It is often called “being sick to your stomach.” Vomiting or throwing-up is forcing the contents of the stomach up through the food pipe (esophagus) and out of the mouth. Common problems that may cause nausea and vomiting include: food allergies, infections of the stomach or bowels, such as the “stomach flu” or food poisoning, leaking of stomach contents (food or liquid) upward (also called gastroesophageal reflux or GERD), medicines or medical treatments, such as cancer chemotherapy or radiation treatment, migraine headaches, morning sickness during pregnancy, seasickness or motion sickness, and severe pain, such as with kidney stones.

### SUMMARY

This section provides a general summary of the disclosure, and is not a comprehensive disclosure of its full scope or all of its features.

One aspect of the disclosure provides a computer-implemented method when executed by data processing hardware of a computing device causes the data processing hardware to perform operations including: receiving, from one or more monitoring devices, patient parameter data of a patient, the patient parameter data collected by the one or more monitoring devices; determining, quantitively assessing, or qualitatively assessing, based on the patient parameter data of the patient, whether the patient is experiencing or about to experience an episode of nausea; and when the patient is experiencing or about to experience an episode of nausea, communicating one or more therapy signals to a receiver device in communication with the computing device, the receiver device configured to contribute to providing nausea therapy to the patient.

Implementations of the disclosure may include one or more of the following optional features. The one or more therapy signals include a nausea therapy delivering device activation signal. The receiver device includes a nausea therapy delivery device associated with the patient. The operation of communicating the one or more therapy signals includes communicating the nausea therapy delivering device activation signal to the nausea therapy delivery device.

In some implementations, the nausea therapy delivering device activation signal when received by the nausea therapy delivery device is configured to activate the nausea therapy delivery device and cause the nausea therapy delivery device to deliver a nausea therapeutic to the patient. The operations further include, after communicating the nausea therapy delivering device activation signal to the nausea therapy delivery device, receiving a confirmation single reporting that the nausea therapy delivering device delivered the nausea therapeutic to the patient. The nausea therapy delivering device includes: an automatic injector; an infusion pump; an inhaler; a transcutaneous patch; a sub-dermal implantable pump; or a small molecule, biologic, cell therapy administrator.

In other implementations, the one or more therapy signals includes a notification signal. The receiver device includes a user computing device. The operation of communicating the one or more therapy signals includes communicating the notification signal to the user computing device. The notification signal when received by the user computing device is configured to display a message on a user interface, the message prompting the patient to self-administer a nausea therapeutic via a nausea therapy delivery device. The operations further include, after communicating the notification signal to the user computing device, receiving a confirmation signal from the user computing device indicating that the patient self-administered the nausea therapeutic via the nausea therapy delivery device.

In yet other implementations, the one or more therapy signals include a report signal. The receiver device includes a healthcare provider computing device associated with a health care provider treating the patient. The operation of communicating the one or more therapy signals includes communicating the report signal to the health care provider computing device, the report signal indicating that the patient is experiencing or about to experience the episode of nausea.

In some configurations, the one or more therapy signals includes a recommendation signal. The receiver device includes a healthcare provider computing device associated with a health care provider treating the patient. The operation of communicating the one or more therapy signals includes communicating the recommendation signal to the health care provider computing device. The recommendation signal when received by the health care provider computing device causing the health care provider computing device to instruct the health care provider to administer a nausea therapeutic to the patient.

In other configurations, the patient parameter data includes one or more physiologic parameters of the patient. The One or more physiologic parameters of the patient includes at least one of: electrodermal activity; skin temperature; core temperature; electrocardiography; blood pressure; heart rate; heartrate variability; sound/voice/speech patterns, or actigraphy.

In yet other configurations, the patient parameter data includes at least one of demographic parameters or anthropometric parameters, or patterns thereof. The at least one of demographic parameters or anthropometric parameters includes at least one of: age; gender; height; weight; body mass index; or disease state.

In one embodiment, the patient parameter data includes at least one of: electrodermal activity; skin temperature; core temperature; electrocardiography; blood pressure; heart rate; heartrate variability; sound/voice/speech patterns, actigraphy, age; gender; height; weight; body mass index; or disease state.

In one embodiment, the patient parameter data includes at least one of: sound/voice/speech patterns, actigraphy, heart rate; heartrate variability, skin conductance such as electrodermal activity and/or temporal changes in Galvanic Skin Response, or patient-reported outcomes.

In one embodiment, the patient parameter data includes at least one of: electrodermal activity, heart rate, heartrate variability, actigraphy, or skin temperature.

In one embodiment, evaluation of emetic events includes at least one of: sound/voice/speech patterns, actigraphy, heart rate; heartrate variability, skin temperature, core temperature, or patient-reported outcomes.

In one embodiment, the patient's parameter data is measured and/or analyzed continuously, on-demand, prodromal, or the onset of symptoms of nausea and/or vomiting.

In one embodiment, at least one of: electrodermal activity, heart rate, actigraphy, or skin temperature is measured and/or analyzed at the prodromal phase.

In some examples, the operation of determining, quantitively assessing, or qualitatively assessing whether the patient is experiencing or about to experience an episode of nausea includes executing a predictive model configured to receive the patient parameter data and generate an indicator score indicating whether the patient is experiencing or about to experience the episode of nausea. The predictive model is trained on a corpus of training parameters associated with training parameter data collected from a first group of subjects who suffer from episodes of nausea and a second group of subjects who do not suffer from episodes of nausea. In one embodiment, an audio recording method could be used as a predictive or indicator monitor.

Another aspect of the disclosure provides a management system including data processing hardware of a computing device and memory hardware of the computing device in communication with the data processing hardware. The memory hardware stores instructions that when executed on the data processing hardware cause the data processing hardware to perform operations including: receiving, from one or more monitoring devices, patient parameter data of a patient, the patient parameter data collected by the one or more monitoring devices; determining, quantitively assessing, or qualitatively assessing based on the patient parameter data of the patient, whether the patient is experiencing or about to experience an episode of nausea; and when the patient is experiencing or about to experience an episode of nausea, communicating one or more therapy signals to a receiver device in communication with the computing device, the receiver device configured to contribute to providing nausea therapy to the patient.

Implementations of the disclosure may include one or more of the following optional features. The one or more therapy signals include a nausea therapy delivering device activation signal. The receiver device includes a nausea therapy delivery device associated with the patient. The operation of communicating the one or more therapy signals includes communicating the nausea therapy delivering device activation signal to the nausea therapy delivery device. The nausea therapy delivering device activation signal when received by the nausea therapy delivery device is configured to activate the nausea therapy delivery device and cause the nausea therapy delivery device (400) to deliver a nausea therapeutic to the patient. The operations further includes, after communicating the nausea therapy delivering device activation signal to the nausea therapy delivery device, receiving a confirmation single reporting that the nausea therapy delivering device delivered the nausea therapeutic to the patient. The nausea therapy delivering device includes: an automatic injector; an infusion pump; an inhaler; a transcutaneous patch; a sub-dermal implantable pump; or a small molecule, biologic, cell therapy administrator.

In some implementations, the one or more therapy signals include a notification signal. The receiver device includes a user computing device. The operation of communicating the one or more therapy signals includes communicating the notification signal to the user computing device. The notification signal when received by the user computing device is configured to display a message on a user interface, the message prompting the patient to self-administer a nausea therapeutic via a nausea therapy delivery device. The operations further includes, after communicating the notification signal to the user computing device, receiving a confirmation signal from the user computing device indicating that the patient self-administered the nausea therapeutic via the nausea therapy delivery device.

In other implementations, the one or more therapy signals include a report signal. The receiver device includes a healthcare provider computing device associated with a health care provider treating the patient. The operation of communicating the one or more therapy signals includes communicating the report signal to the health care provider computing device, the report signal indicating that the patient is experiencing or about to experience the episode of nausea.

In yet other implementations, the one or more therapy signals include a recommendation signal. The receiver device includes a healthcare provider computing device associated with a health care provider treating the patient. The operation of communicating the one or more therapy signals includes communicating the recommendation signal to the health care provider computing device, the recommendation signal when received by the health care provider computing device causing the health care provider computing device to instruct the health care provider to administer a nausea therapeutic to the patient.

In some configurations, the patient parameter data includes one or more physiologic parameters of the patient. One or more physiologic parameters of the patient includes at least one of: electrodermal activity; skin temperature; core temperature; electrocardiography; blood pressure; heart rate; heartrate variability; sound/voice/speech patterns, or actigraphy.

In other configurations, the patient parameter data includes at least one of demographic parameters or anthropometric parameters. The at least one of demographic parameters or anthropometric parameters includes at least one of: age; gender; height; weight; body mass index; or disease state.

In yet other configurations, the operation of determining whether the patient is experiencing or about to experience an episode of nausea includes executing a predictive model configured to receive the patient parameter data and generate an indicator score indicating whether the patient is experiencing or about to experience the episode of nausea. The predictive model is trained on a corpus of training parameters associated with training parameter data collected from a first group of subjects who suffer from episodes of nausea and a second group of subjects who do not suffer from episodes of nausea.

Another aspect of the disclosure provides a system/method for managing nausea and vomiting for a patient. As used herein, managing or management of nausea and vomiting refers to tracking of the symptoms/clinical manifestations, quantitatively or qualitatively assessing emetic events and/or episodes, predicting nausea and/or vomiting, and predicting the prodromal phase of nausea and/or vomiting. The system/method includes at least one computing device and one monitoring device. At least one sign, or surrogate biomarker can be used by at least one monitoring device (e.g., a stationary patient monitor, portable patient monitor, Holter-style monitor, wearable monitor, or an audio recording device, etc.). The computing device connects to the monitoring device and receives information from the monitoring device and processes the information to evaluate one or more nausea and/or vomiting symptom levels or predicts the vomiting episodes. In one embodiment, the monitor device is an audio recording device.

In one embodiment, the disclosure provides a method for treating a disease or preventing a disease progression in a subject comprising:
(a) a monitor device for tracking one or more symptoms, clinical manifestations, or therapy signals of the disease;
(b) a computing device for analyzing the symptoms, clinical manifestations, or therapy signals of the disease and to determining whether the subject is experiencing or about to experience an episode of the disease;
(c) a receiver device in communication with the computing device, and optionally; and
(d) a therapy delivery device for enabling administration of a therapeutic agent for preventing the disease to the subject in need thereof; wherein the disease is nausea, retch, and/or vomiting.

In one embodiment, the method monitors the patient's status either continuously, on-demand, prodromal, or the onset of symptoms of nausea and/or vomiting.

In one embodiment, the method comprising monitoring, computing, assessing, or receiving input of one or more physiologic, demographic, and/or anthropometric characteristics.

In one embodiment, the physiologic, demographic, and/or anthropometric characteristics comprises: electrodermal activity, temperature, electrocardiography, blood pressure, heart rate, heartrate variability, actigraphy, age, gender, height, weight, body mass index, sleep activity, diagnosis of inflammatory bowel disease, ulcerative colitis, or cyclic vomiting syndrome.

In one embodiment, the method comprising identifying, counting, or quantitatively assessing the number of emetic events.

In one embodiment, the emetic events is nausea, retch, or vomiting.

In one embodiment, the therapy agent is an agent for treating or preventing nausea or vomiting.

In one embodiment, the delivery device comprises at least one of: an automatic injector, an infusion pump, an inhaler, a trans-cutaneous patch, a sub-dermal implantable pump, or a small molecule, biologic, cell therapy administrator.

In one embodiment, the method comprises a computer-implemented method when executed by data processing hardware of a computing device causes the data processing hardware to perform operations including:
(1) receiving patient parameter data of a patient collected by the one or more monitoring devices;
(2) determining, based on the patient parameter data of the patient, whether the patient is experiencing or about to experience an episode of nausea and/or vomiting; and
(3) when the patient is experiencing or about to experience an episode of nausea or vomiting, communicating one or more therapy signals to a receiver device in communication with the computing device, the receiver device configured to contribute to providing nausea therapy to the patient.

In one embodiment, the method includes a therapy delivering device activation signal.

In one embodiment, the receiver device includes a therapy delivery device associated with the patient.

In one embodiment, the operation of communicating the symptoms, clinical manifestations, or therapy signals includes communicating the therapy delivering device activation signal to the therapy delivery device.

In one embodiment, the therapy delivering device activation signal when received by the therapy delivery device is configured to activate the therapy delivery device and cause the therapy delivery device to deliver a therapeutic agent to the patient.

In one embodiment, the therapy agent is an agent for treating or preventing nausea or vomiting.

In one embodiment, the method further comprises, after communicating the therapy delivering device activation signal to the therapy delivery device, receiving a confirmation single reporting that the therapy delivery device delivered the therapeutic agent to the patient.

In one embodiment, the therapy delivery device includes an automatic injector; an infusion pump; an inhaler; a transcutaneous patch; a sub-dermal implantable pump; or a small molecule, biologic, cell therapy administrator.

In one embodiment, the therapy signals include a notification signal; wherein the receiver device includes a user computing device, wherein the operation of communicating the therapy signals includes communicating a notification signal to a user computing device.

In one embodiment, the notification signal when received by the user computing device is configured to display a message on a user interface, wherein the message prompts the patient to self-administer a therapeutic agent via a therapy delivery device.

In one embodiment, the method further includes, after communicating the notification signal to the user computing device, receiving a confirmation signal from the user computing device indicating that the patient self-administered the therapeutic agent via the nausea therapy delivery device.

In one embodiment, the therapy signals include a report signal, wherein the receiver device includes a healthcare provider computing device associated with a health care provider treating the patient, wherein the operation of communicating the therapy signals includes communicating a report signal to a health care provider computing device.

In one embodiment, the report signal indicates that the patient is experiencing or about to experience the episode of nausea.

In one embodiment, the therapy signals includes a recommendation signal, wherein the recommendation signal when received by the health care provider computing device causes the health care provider computing device to instruct the health care provider to administer a therapeutic agent to the patient.

In one embodiment, the patient parameter data includes one or more parameters of the patient comprising electrodermal activity, skin temperature, core temperature, electrocardiography, blood pressure, heart rate, heartrate variability, age, gender, height, weight, body mass index, or disease state.

In one embodiment, the operation of determining whether the patient is experiencing or about to experience an episode of nausea or vomiting includes executing a predictive model configured to receive the patient parameter data and generate an indicator score indicating whether the patient is experiencing or about to experience the episode.

In one embodiment, the predictive model is trained on a corpus of training parameters associated with training parameter data collected from a first group of subjects who suffer from episodes of nausea or vomiting and a second group of subjects who do not suffer from episodes of nausea or vomiting.

In one embodiment, the method provides a management system including data processing hardware of a computing device and memory hardware of the computing device in communication with the data processing hardware.

In one embodiment, the memory hardware stores instructions that when executed on the data processing hardware causes the data processing hardware to perform operations including: receiving, from one or more monitoring devices, patient parameter data of a patient, the patient parameter data collected by the one or more monitoring devices; determining, based on the patient parameter data of the patient, whether the patient is experiencing or about to experience an episode of nausea; and when the patient is experiencing or about to experience an episode of nausea, communicating one or more therapy signals to a receiver device in communication with the computing device, the receiver device configured to contribute to providing nausea therapy to the patient.

In one embodiment, the method includes one or more of the following optional features:
(1) the one or more therapy signals include a therapy delivering device activation signal;
(2) the receiver device includes a therapy delivery device associated with the patient who is experiencing or about to experience an episode of nausea and/or vomiting;
(3) the operation of communicating the therapy signals includes communicating the therapy delivery device activation signal to the therapy delivery device; and
(4) the therapy delivery device activation signal when received by the therapy delivery device is configured to activate the therapy delivery device and cause the therapy delivery device (400) to deliver a therapeutic agent to the patient; wherein the therapy delivery device includes an automatic injector, an infusion pump, an inhaler, a transcutaneous patch, a sub-dermal implantable pump, or a small molecule, biologic, cell therapy administrator, and optionally the method further includes (5) receiving a confirmation single reporting that the therapy delivering device delivered the therapeutic agent to the patient.

In one embodiment, the system/method further includes at least one therapeutic agent capable of either preventing or treating nausea and/or vomiting.

In one embodiment, the system/method measures or receives input of one or more physiologic, demographic, and/or physiologic, demographic and anthropometric characteristics, such as: electrodermal activity (EDA), temperature (skin and/or core), electrocardiography (ECG), blood pressure (BP), heart rate (HR), heartrate variability (HRV), actigraphy, age, gender, height, weight, body mass index (BMI), sleep activity, diagnosis of inflammatory bowel disease (IBD), ulcerative colitis (UC), cyclic vomiting syndrome (CVS), and patient reported outcomes/symptoms, etc. Preferably EDA, specifically temporal changes in Galvanic Skin Response (GSR), HR, specifically heart rate variability (HRV) and sleep activity specifically sleep quality which is reported by patient, or sleep architecture measured by a device are used. The system/method monitors the patient's status, continuously or on-demand, the prodromal or the onset of symptoms of nausea and/or vomiting. The system also provides recommendations for administration of a therapeutic agent (e.g., small molecule, biologic, cell therapy, etc.) as well as a route of administration (e.g., oral (PO), transcutaneous injection (TC), intravenous therapy (IV), intramuscular injection (IM), etc.). In another embodiment, the system/method calculates a patient-specific risk score indicating the patient's risk of nausea and/or vomiting.

In another embodiment, the system/method identify and/or count the number of emetic (vomit and retch) events.

In another embodiment, the system/method quantitatively assess emetic events and/or episodes. In another embodiment, the system/method identify and/or evaluate the severity of emetic (vomit and retch) events.

In another embodiment, the emetic events and/or episodes are nausea, retch, and vomiting.

In another embodiment, the system/method includes a delivery device (e.g. automatic injector, infusion pump, inhaler, trans-cutaneous patch, sub-dermal implantable pump, etc.) in communication with the monitoring device, either wired or wirelessly. In this embodiment, the monitoring device activates the delivery device as needed to administer the therapeutic agent to the patient.

In another embodiment, based on an analysis of the patient's physiological parameters/variables, such as HR, temperature, or other indicators of dehydration, the system/method indicates a need for additional treatment. Additional or alternatively, based on such analysis, the system/method may recommend transporting the patient to a medical facility,

The system/method detects, measures, and/or quantifies physiological parameters and changes in those parameters that are indicative or predictive of nausea and/or vomiting. The system/method calculates a trend and/or variability in the data collected.

In one embodiment, the system/method employs a moving average function for averaging data collected during a period of time or window, such as 1 minute, 2 minutes, or 10 minutes to smooth and represent the trend of the measured parameter(s).

In another embodiment, the system/method employs a digital filter to smooth and represent the trend in the dataset. The digital filter may be one or more of the following: Parks-McClellan filter, Chebyshev filter, Butterworth filter, Bessel filter, elliptic filter, constant-k filter, m-derived filter, special filter, top-hat filter, or a Fourier-transform-based filter, where a window for the filter may be 1 minute, 2 minutes, 10 minutes, or up to the length of the dataset.

In one embodiment, the system/method employs at least one technique of calculating the variability on the measured parameter(s) over various data windows, where the method is applied to at least one of a raw dataset, a smoothed dataset, or multiple smoothed datasets. The technique used to calculate the variability can be one or more of variance, standard deviation, kurtosis, min-max, interquartile range (IQR), etc.

In one embodiment, the system/method uses either supervised or unsupervised machine learning algorithm (e.g., naive Bayes classifier, k-means clustering, linear/logistic regression, random forests, decision trees, etc.) trained on the patient's own data and/or a population dataset to determine which physiologic parameters to monitor as well as what are the specific thresholds that are indicative/predictive of nausea and/or vomiting. Such indication or prediction may be based on a correlation between a physiological parameters of a patient and outcomes (symptoms) reported by the patient, for instance, measured skin turgor.

In one embodiment, a wrist-worn wearable device with an input of the patient's age, gender, height, and weight and continuously measuring EDA and HR is combined with an IM auto-injector prefilled with a therapeutic agent, medicament or drug known and/or effective for treating or preventing nausea and/or vomiting, or known and/or effective for reducing one or more symptoms associated with nausea and/or vomiting. The system/method continuously calculates mean, median, standard deviation, and z-score of EDA and HRV over a 1-minute window and using those values, along with a multinomial logistic regression model trained on a large patient population dataset, continuously calculates a patient-specific risk-score. If the risk score exceeds predefined limits, the system/method triggers a warning to the patient to use the auto injector and administer the therapeutic agent to prevent the onset of vomiting.

In one embodiment, the system/method tracks the presence of potential adverse events (AEs)/tolerability issues.

The details of one or more implementations of the disclosure are set forth in the accompanying drawings and the description below. Other aspects, features, and advantages will be apparent from the description and drawings, and from the claims.

### BRIEF DESCRIPTION OF DRAWINGS

FIG. 1 is a schematic view of an example system for managing nausea and vomiting for a patient.

FIG. 2 is a schematic view of an example patient device used in managing nausea and vomiting for a patient.

FIG. 3A is a schematic view of an example monitor used in managing nausea and vomiting for a patient.

FIG. 3B is a schematic view of an example monitor used in managing nausea and vomiting for a patient.

FIG. 4A is the actigraphy activity data collected from a patient in the Study.

FIG. 4B is the 1-minute SD of actigraphy data collected from a patient in the Study.

FIG. 4C is the electrodermal activity data collected from a patient in the Study.

FIG. 4D is the 1-minute SD of electrodermal activity data collected from a patient in the Study.

FIG. 4E is the temperature collected from a patient in the Study.

FIG. 4F is the 1-minute SD of temperature collected from a patient in the Study.

FIG. 4G is the heart rate of collected from a patient in the Study.

FIG. 5 is a flowchart of an example arrangement of operations for a method of managing vomiting and nausea for a patient by using the system of FIG. 1.

FIG. 6 is a schematic view of an example of a computing device that may be used to implement the systems and methods described herein.

Like reference symbols in the various drawings indicate like elements.

### DETAILED DESCRIPTION

Cyclic vomiting syndrome (CVS) is a disorder that causes recurrent episodes of nausea, vomiting, and tiredness (lethargy). This condition is diagnosed most often in young children, but it can affect people of any age. The episodes of nausea, vomiting, and lethargy last anywhere from an hour to ten days. An affected person may vomit several times per hour, potentially leading to a dangerous loss of fluids (dehydration). Additional symptoms can include unusually pale skin (pallor), abdominal pain, diarrhea, headache, fever, and an increased sensitivity to light (photophobia) or to sound (phonophobia). In most affected people, the signs and symptoms of each attack are quite similar. These attacks can be debilitating, making it difficult for an affected person to go to work or school.

Episodes of nausea, vomiting, and lethargy can occur regularly or apparently at random, or can be triggered by a variety of factors. The most common triggers are emotional excitement and infections. Other triggers can include periods without eating (fasting), temperature extremes, lack of sleep, overexertion, allergies, ingesting certain foods or alcohol, and menstruation.

If the condition is not treated, episodes usually occur four to twelve times per year. Between attacks, vomiting is absent, and nausea is either absent or much reduced. However, many affected people experience other symptoms during and between episodes, including pain, lethargy, digestive disorders such as gastroesophageal reflux and irritable bowel syndrome, and fainting spells (syncope). People with cyclic vomiting syndrome are also more likely than people without the disorder to experience depression, anxiety, and panic disorder. Therefore, it is desirable to have a solution for managing nausea and vomiting for a patient.

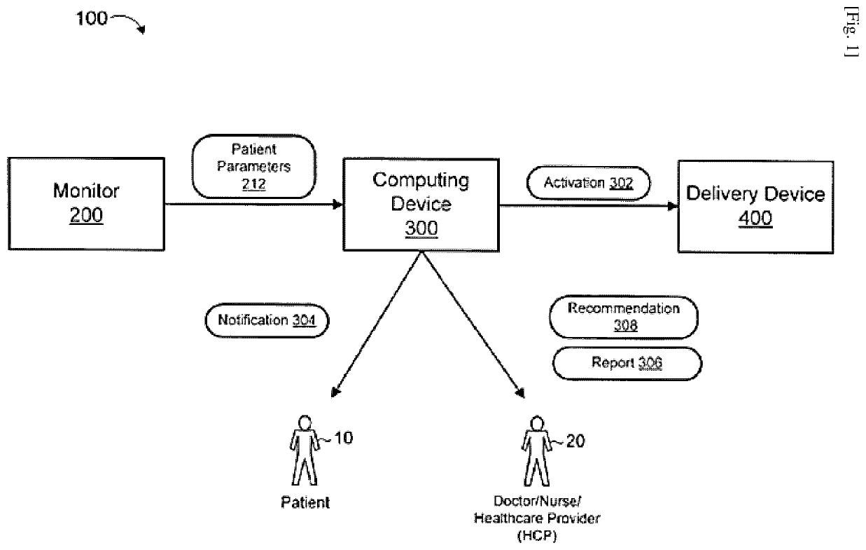

Referring to FIG. 1, in some implementations, a patient 10 uses a management system 100 to track symptoms or clinical manifestations of a disease, to quantitatively assess emetic events and/or episodes, or to predict episodes of nausea and/or vomiting. The management system 100 includes one or more patient device(s) 200, a computing device 300, and a delivery device 400. The monitoring devices(s) 200 measure the patient's 10 physiologic parameters, such as electrodermal activity (EDA), temperature (skin and/or core), electrocardiography (ECG), blood pressure (BP), heart rate (HR), heartrate variability (HRV), and actigraphy. The monitoring devices(s) 200 also collects demographic and/or anthropometric parameters that are inputted by the patient 10, such as their age, gender, height, weight, body mass index (BMI). The monitoring devices(s) 200 may also other collect information related to a disease state, such as a diagnosis of inflammatory bowel disease (IBD), ulcerative colitis (UC), and cyclic vomiting syndrome (CVS), to name a few examples. The monitoring devices(s) 200 communicates the measured physiologic parameters, the inputted demographic/anthropometric parameters, and other information such as sound, voice, or speech patterns, collectively referred to as patient parameters 212, to the computing device 300. A patient monitoring device 200 may include a device capable of recording audio corresponding to speech spoken by the patient 10. As such, recorded audio including speech spoken by the patient 10 may provide patterns of sounds in the patient's speech that may convey information related to a disease state.

The computing device 300 analyzes the patient parameters 212 and determines whether the patient 10 is experiencing or is about to experience an episode of nausea and/or vomiting. In some implementations of the computing device 300, the analysis includes identifying changes in the patient parameters 212 that are indicative or predictive of the patient 10 experiencing or is about to experience an episode of nausea and/or vomiting. For example, the computing device 300 calculates a trend and/or variability in the patient parameters 212 that correspond to the patient 10 reporting that they are experiencing nausea and/or vomiting. Other implementations of the computing device 300 use a machine learning algorithm trained on a large population dataset to determine which of the patient parameters 212 to monitor as well as what are the specific thresholds that are indicative or predictive of patient 10 experiencing or is about to experience an episode of nausea and/or vomiting. The computing device 300 in some instances may take into account patient parameters corresponding to a previous determination in order to make a current determination of whether the patient 10 is experiencing or is about to experience an episode of nausea and/or vomiting.

When the computing device 300 determines the patient 10 is experiencing or is about to experience an episode of nausea and/or vomiting, the computing device 300 activates the delivery device 400 (by way of an activation signal 302) enabling administration of a therapeutic agent capable of treating nausea and/or vomiting. Examples of the delivery device 400 include an automatic injector, infusion pump, inhaler, transcutaneous patch, sub-dermal implantable pump, or other device capable of administering a small molecule, biologic, cell therapy, and the like. The computing device 300 may receive a confirmation from the delivery device 400 reporting that the delivery device 400 is successfully activated and ready for the patient 10 to use.

In one embodiment, determining whether a patient 10 is experiencing or about to experience an episode of nausea is based quantitatively assessing or qualitatively assessing the patient parameter data 212.

The computing device 300 notifies the patient 10 (by way of a notification 304) to use the activated delivery device 400 to administer the therapeutic agent. The computing device 300 may also receive a confirmation(s) from the patient 10 acknowledging that the patient 10 received the notification 304 and/or reporting that the patient 10 successfully administered the therapeutic agent. The computing device 300 may also notify the patient's 10 healthcare provider (HCP) 20, who may be a doctor, a nurse, or a clinician, (by way of a report 306) that the patient 10 is experiencing or is about to experience an episode of nausea and/or vomiting. For instance, a HCP computing device 21 associated with the HCP 20 may receive the notification 304. The HCP computing device 21 may be referred to as a “receiver device” in communication with the computing device 300.

Upon determining the patient 10 is experiencing or is about to experience an episode of nausea and/or vomiting, some examples of the computing device 300 make a recommendation 308 for administrating a therapeutic agent as well as a route of administration, e.g., oral (PO), transcutaneous injection (TC), intravenous therapy (IV), intramuscular injection (IM), etc. Other examples of the computing device 300 make a recommendation 308 to transport the patient 10 to a medical facility, such as an emergency room, urgent care, specialized in- or out-patient clinic, or the like. In these examples, the computing device 300 communicates the recommendation 308 to the healthcare provider 20. The patient 10, the HCP 20, the patient device(s) 200, the computing device 300, and/or the delivery device 400 may exchange other messages not shown in the figure.

In some implementations, the forgoing messages among the patient 10, the HCP 20, the patient device(s) 200, computing device 300, and the delivery device 400 may be communicated via a communications network such as the Internet or a local area network. Such network may provide access to cloud computing resources that allow some functions of the patient device(s) 200, the computing device 300, and/or the delivery device 400 to be performed on or distrusted to one or more remote devices, i.e., at location(s) different than a physical location of the patient 10.

The network 30 may include any type of network that allows sending and receiving communication signals/messages, such as a wireless telecommunication network, a cellular telephone network, a time division multiple access (TDMA) network, a code division multiple access (CDMA) network, Global system for mobile communications (GSM), a third generation (3G) network, fourth generation (4G), fifth generation (5G) network, a satellite communications network, and other communication networks. The network 30 may include one or more of a Wide Area Network (WAN), a Local Area Network (LAN), and a Personal Area Network (PAN). In some examples, the network 30 includes a combination of data networks, telecommunication networks, and a combination of data and telecommunication networks. In some examples, the network 30 provides access to cloud computing resources, which may be elastic/on-demand computing and/or storage resources available over the network 30. The term ‘cloud’ services generally refers to a service performed not locally, but rather delivered from one or more remote devices accessible via one or more networks 30.

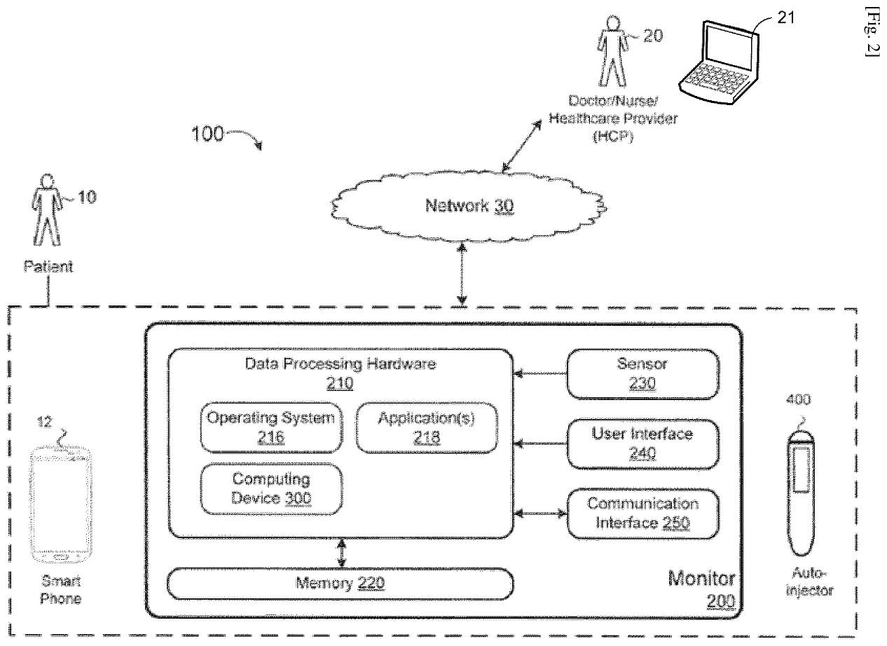

FIG. 2 shows an example of the monitoring device 200, the computing device 300, and delivery device 400 used by the patient 10 to manage their nausea and/or vomiting. The patient 10 may be experiencing or is about to experience an episode of nausea and/or vomiting because they are taking chemotherapy medication or are suffering from a condition, such as inflammatory bowel disease (IBD), ulcerative colitis (UC), and cyclic vomiting syndrome (CVS). In some cases, the patient 10 may be suffering from chronic nausea and vomiting that cannot be explained. In other cases, the patient 10 may be nausea and feel like they want to vomit because they are experiencing vertigo or motion sickness.

In the example shown, the monitoring device 200 incorporates the computing device 300 as an application and executes the functionality of the computing device 300. The monitoring device 200 may be a stationary patient monitor, a portable patient monitor, a Holter-style monitor, a wearable medical device, or an audio recording device, just to name a few examples. In general, the monitoring device 200 includes data processing hardware 210, memory 220, sensor(s) 230, a user interface 240, and a communication interface 250. The monitoring device 200 may include additional components not shown in the figure.

The data processing hardware 210 includes a processor that executes computer-executable instructions stored in the memory 220 to provide the functionality of the computing device 300. Additionally, the processor may execute an operating system 216 and other application(s) 218, all of which may be implemented as computer-readable instructions. The data processing hardware 210 can include more processors that execute the computer-executable instructions stored in the memory 220. In implementations in which the monitoring device 200 includes more than one processor, the processors can execute the functions of the monitoring device 200, including the functionality of the computing device 300, in a distributed or individual manner.

The memory 220 includes one or more computer-readable mediums (e.g., hard disk drives, solid state memory drives, and/or flash memory drives). The memory 220 can store any suitable data that is used by the operating system 216, the application(s) 218, and the computing device 300.

The sensor 230 provides the patient's 10 physiologic parameters to the data processing hardware 210 where the computing device 300 analyses the physiologic parameters to determine whether the patient 10 is experiencing or is about to experience an episode of nausea and/or vomiting. The sensor 230 includes one or devices configured to detect, measure, and/or quantify physiological parameters of the patient 10, including but not limited to, electrodermal activity (EDA), temperature (skin and/or core), electrocardiography (ECG), blood pressure (BP), heart rate (HR), heartrate variability (HRV), and actigraphy. Examples of the sensor 230 include temperature sensor, blood glucose sensor, blood oxygen sensor, ECG sensor, image sensor, motion sensor, inertial sensor, pressure sensor, photoplethysmography sensor, and the like. Furthermore, the sensor 230 may be wearable, implantable, invasive, non-invasive, or ingestible.

The user interface 240 receives demographic and/or anthropometric parameters that are inputted by the patient 10, such as their age, gender, height, weight, and body mass index (BMI), and provides the inputted parameters to the data processing hardware 210. Additionally or alternatively, the user interface 240 provides the inputted parameters to the memory 220 to be stored and later retrieved by the data processing hardware 210. At the data processing hardware 210, the computing device 300 analyses the inputted parameters together with the physiologic parameters from the sensors 230 to determine whether the patient 10 is experiencing or is about to experience an episode of nausea and/or vomiting. In some implementations, the user interface 240 may also receive information related to a disease state, such as a diagnosis of inflammatory bowel disease (IBD), ulcerative colitis (UC), and cyclic vomiting syndrome (CVS), to be analyzed with the other patient parameters. The user interface 240 can also provide output to the patient 10, such the notification 304 (see, e.g., FIG. 1).

The user interface 240 includes devices, including but not limited to, a touchscreen, a display, a QWERTY keyboard, a numeric keypad, a touchpad, a microphone, and/or speakers. Some examples of the user interface 240 include the patient's 10 smart phone 12. In these examples, the patient 10 uses their smart phone 12 to enter in demographic and/or anthropometric parameters.

The communication interface 250 includes one or more components that are configured to communicate with the patient 10 (by way of their smart phone 12), the HCP 20 (by way of the network 30), and/or the delivery device 400. The communication interface 260 can include one or more transceivers for performing wired or wireless communication. Examples of the communication interface 260 can include, but are not limited to, a transceiver configured to perform communications using the IEEE 802.11 wireless standard, an Ethernet port, a wireless transmitter, and a universal serial bus (USB) port.

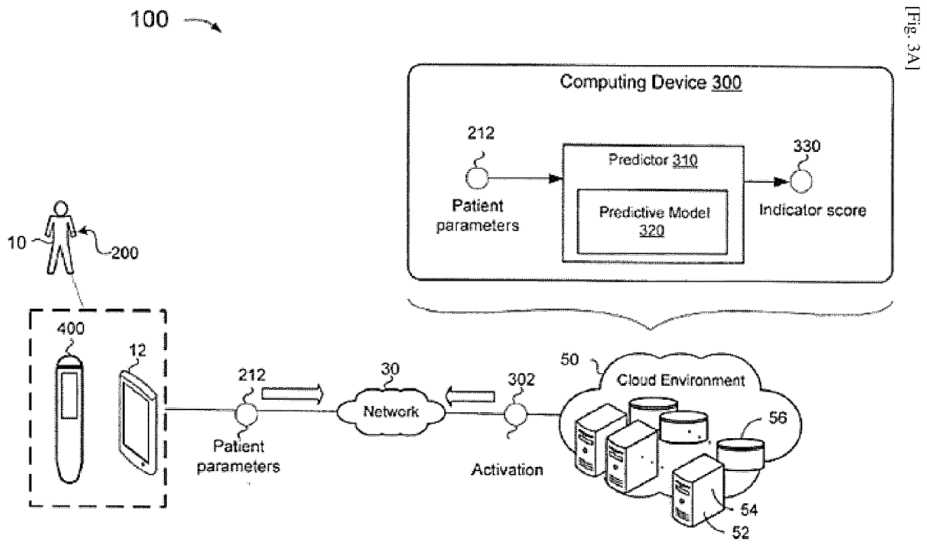

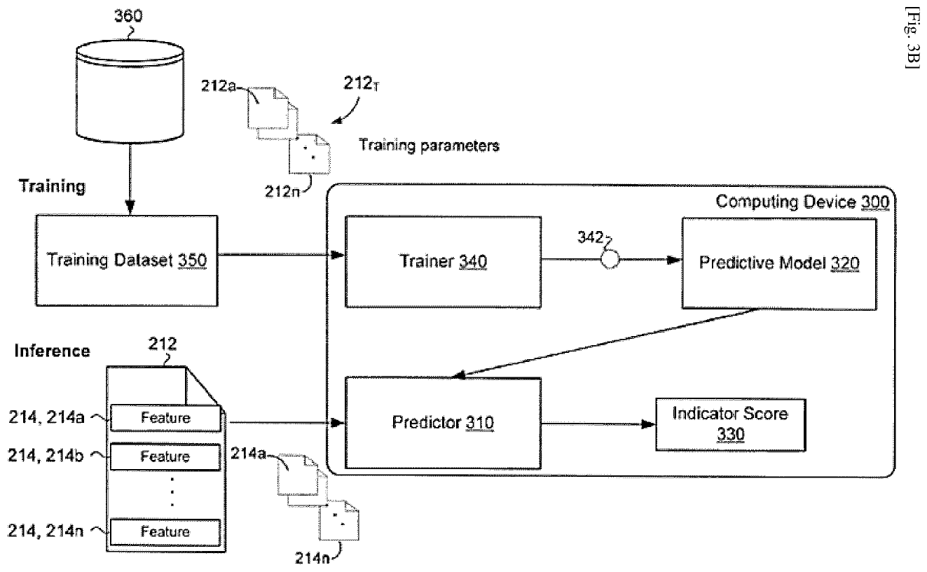

FIG. 3A shows an example of the management system 100 for predicting episodes of nausea and/or vomiting. The management system 100 includes a monitoring device 200, which in the example shown is worn by a patient 10 around their wrist. The monitoring device 200 detects and measures physiologic parameters of the patient 10. A user computing device 12 (e.g., smart phone) collects the physiologic parameters sensed by the monitoring device 200 along with demographic and/or anthropometric parameters that are inputted by the patient 10 as patient parameters 212 over a network 30, for example, to a remote system 50. The user computing device 12 may correspond to a “receiver device” in communication with the computing device 300. The remote system 50 may be a distributed system (e.g., a cloud or edge computing environment) having scalable/elastic computing resources 52, 54 (e.g., data processing hardware) and/or storage resources 56 (e.g. memory hardware).

In some implementations, the remote system 50 executes a computing device 300 configured to receive patient parameters 212 from the patient 10 and generate an indicator score 330 based on the received parameters. The indicator score 330 indicates a likelihood of the patient 10 is experiencing or is about to experience an episode of nausea and/or vomiting. When the indicator score 330 indicates that the patient 10 is likely experiencing or is likely to experience an episode of nausea and/or vomiting, the computing device 300 outputs an activation signal 302 over the network 30. The activation signal 302 activates a delivery device 400 thereby enabling the patient 10 to use the delivery device 400 to self-administer a therapeutic agent capable of treating nausea and/or vomiting.

In other implementations of the management system 100, the computing device 300 continuously calculates an indicator score 330 as a patient-specific risk-score for a patient 10. If the patient-specific risk-score exceeds one or more pre-defined limits, the management system 100 triggers a warning to the patient 10 to use an auto-injector and administer a therapeutic agent to prevent the onset of nausea and/or vomiting.

Referring to FIG. 3B, the computing device 300 includes a predictor 310 that uses a predictive model 320 to generate the indicator score 330. The predictive model 320 may be trained by a trainer 340 based on a training dataset 350. In some examples, the predictor 310 uses a predictive model 320 that is configured to receive a plurality of features 214, 214a-n associated with the patient parameters 212 as feature inputs 214. The plurality of features 214a-n includes features 214 related to measurements of electrodermal activity, heart rate, and heartrate variability, for example.

In some implementations, the predictive model may make use of audio data that captures sounds around the patient (including the patient's own vocalizations). In the preferred implementation, audio data will be acquired using an application running on a commercial smartphone. However, in other implementations, audio data may be acquired using a specialized systems worn by the patient, set up in a fixed location in the subject's environment, or a combination of wearable and fixed sensors. These systems may employ either a single microphone or an array of microphones. In some implementations, these sensors may be affixed directly to the subject's skin (in the form of a patch device).

In some implementations, the acquired audio data will be processed using digital signal processing techniques which will filter out background noise and detect audio events of interest, then extract useful features of the audio signal (audio signature). The derived features may include time-domain features capturing the amplitude or frequency content of the signal, outputs of time-frequency analysis (i.e. short-time Fourier transform or wavelet transform) capturing changes in the signal over time, or custom features derived from frequency domain analysis, such as power ratios between different frequency bands. These derived features will then be processed using machine learning models based on previously acquired training data that will classify emesis (vomit or retch) sounds for input to the predictive model.

In an alternative implementation to the one described above (which uses hand-crafted features), audio analysis may be accomplished with end-to-end deep learning systems, which learn distinctive features directly from either raw audio or spectral representations of audio (for example spectrograms or Mel spectra). These end-to-end systems may also be combined with more standard digital signal processing methods for initial data cleaning.

In the preferred implementation, audio processing results (“audio signature) will be combined with additional data (for example EDA or HRV data) to generate predictive model. However, in some implementations, audio data may be employed without being combined with other data sources.

In some implementations, the predictive model 320 is trained on the training dataset 350, which is obtained from a data store 360 residing on the storage resources 56 of the distributed system 50, or may reside at some other remote location in communication with the distributed system 50. The training dataset 350 includes a corpus of training parameters 212_{T} collected from a first group of subjects who suffer from episodes of nausea and/or vomiting, such as chemotherapy induced nausea and vomiting, and from a second group of subjects who are healthy (i.e., who do not suffer from episodes of nausea and/or vomiting). Each training parameters 212_{T} includes a corresponding plurality of features. For example, each training parameters 212_{T} includes features related to electrodermal activity, heart rate, and heartrate variability.

In the example shown, the trainer 340 receives the training dataset 350 for training the predictive model 320. Based on the training dataset 350, the trainer 340 models score parameters 342 to train the predictive model 320. The predictor 310 uses the trained predictive model 320 during inference for determining the indicator scores 330 for patient parameters 212. As such, the predictive model 320 is trained to determine indicator scores 330 using the training dataset 350 associated with the corpus of training parameters 212_{T}, each of which includes a corresponding plurality of features.

In some implementations, the predictive model 320 is a multinomial logistic regression model trained on a large patient population dataset. Other examples of the computing device 300 use either supervised or unsupervised machine learning algorithm (e.g., naive Bayes classifier, k-means clustering, linear/logistic regression, random forests, decision trees, etc.) based on the patient's own patient parameters (e.g., physiologic, demographic and/or anthropometric parameters) and/or a population dataset to determine which patient parameters to monitor as well as what are the specific thresholds that are indicative or predictive of nausea and/or vomiting. For some applications, the performance of the predictive model 320 may be evaluated by splitting the dataset into a separate test and training set. A cross-validation technique, such as k-fold cross-validation or leave-one-out is then sued on the training set to find the “optimal” set of hyperparameters for the predicative model. The independent test set is then used to obtain an unbiased estimate of the performance of the predictive model 320.

For some applications, the computing device 300 performs an initial processing step or steps on the patient parameters 212 prior to predicting an episode of nausea and/or vomiting. In these applications, it may be said that the computing device 300 combines digital signal processing and machine learning to generate an indicator score from the patient parameters 212. For example, the computing device 300 applies a moving average function or filter to smooth and represent a trend in patient parameters 212 collected over a period of time from the patient 10. For the sake of explanation, patient parameters collected over a period of time are referred to as a “dataset” and a period of time over which a dataset is collected is referred to as a “window”.

Generally, a moving average means a real time average on a certain number (average number) of input values. For instance, in an example case in which the average number is set to four, in order to calculate the moving average, an average on a recent four input values is calculated at every sample point. A moving average filter/function uses the calculated moving average to perform a “noise” filtering operation. That is, the moving average filter accumulates input values by the average number and outputs an average of the sum of the accumulated input values at every sampling point. Smoothing and representing a trend in this manner may lead to more accurate prediction of nausea and/or vomiting. In some examples, the computing device 300 uses a moving average function with a window (e.g. 1 minute, 2 minutes, or 10 minutes) to smooth and represent a trend of the patient parameters 212.

In other examples, the computing device 300 smooths and represents a trend in a dataset collected from a patient 10 by applying a digital filter to the dataset. In these examples, the digital filter identifies and removes high-frequency fluctuations or “noise” from the dataset. Smoothing and representing a trend in this manner may lead to more accurate prediction of nausea and/or vomiting. Examples of the digital filter include, but are not limited to, Parks-McClellan filter, Chebyshev filter, Butterworth filter, Bessel filter, elliptic filter, constant-k filter, m-derived filter, special filter, top-hat filter, and Fourier-transform-based filter.

The initial processing performed by the computing device 300 may also include calculating a variability of the patient parameters 212 over various data windows. The operation is applied to at least one of a raw dataset (i.e., dataset that has not been smoothed), smoothed dataset, and multiple smoothed datasets. The operation of calculating patient parameter variability can include calculating a variance, standard deviation, kurtosis, min-max, and interquartile range (IQR) to name a few. In a convenient example of the management system 100, the computing device 300 continuously calculates mean, median, standard deviation, and z-score of EDA and HRV parameters collected from patient 10. Variability in the patient's 10 EDA and HRV parameters are calculated over a 1-minute window.

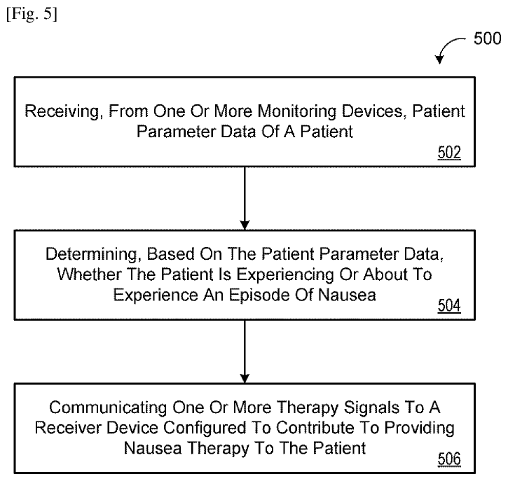

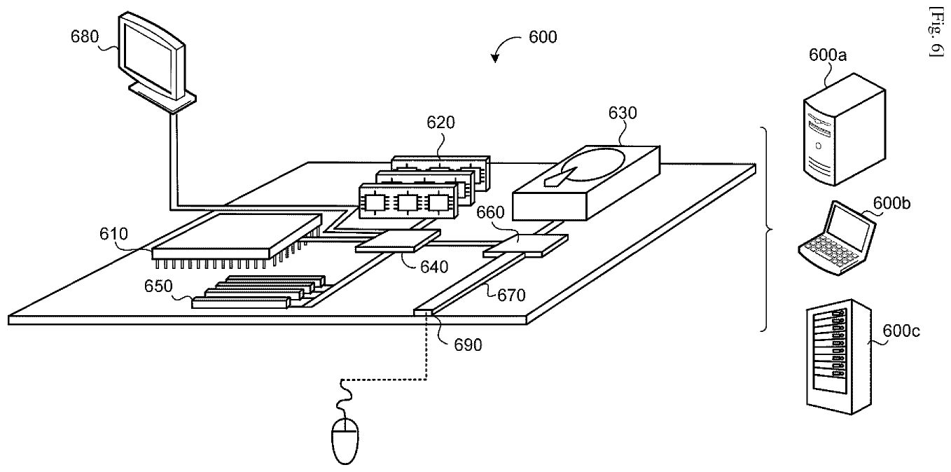

FIG. 5 is a flowchart of an example arrangement of operations for a method 500 of managing nausea and/or vomiting. Data processing hardware 610 (FIG. 6) of a computing device 300 may execute instructions stored on memory hardware 620 (FIG. 6) of the computing device 300 that causes the data processing hardware 610 to perform the operations for the method 500. At operation 502, the method 500 includes receiving, from one or more monitoring devices 200, patient parameter data 212 of a patient 10. The patient parameter data 212 is collected by the one or more monitoring devices (200). At operation 504, the method 500 also includes determining, based on the patient parameter data 212 of the patient 10, whether the patient 10 is experiencing or about to experience an episode of nausea. At operation 506, when the patient 10 is experiencing or about to experience an episode of nausea, the method 500 also includes communicating one or more therapy signals 302, 304, 306, 308 to a receiver device 12, 21, 400 in communication with the computing device. The receiver device 12, 21, 400 configured to contribute to providing nausea therapy to the patient 10.

FIG. 6 is schematic view of an example computing device 600 that may be used to implement the systems and methods described in this document. The computing device 600 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 600 includes a processor 610, memory 620, a storage device 630, a high-speed interface/controller 640 connecting to the memory 620 and high-speed expansion ports 650, and a low speed interface/controller 660 connecting to a low speed bus 670 and a storage device 630. Each of the components 610, 620, 630, 640, 650, and 660, are interconnected using various busses, and may be mounted on a common motherboard or in other manners as appropriate. The processor 610 can process instructions for execution within the computing device 600, including instructions stored in the memory 620 or on the storage device 630 to display graphical information, including, for example, the notification 304, the report 306 and the recommendation 308 described above in FIG. 1; and the indicator score 330 described above in FIG. 3A and FIG. 3B, for a graphical user interface (GUI) on an external input/output device, such as display 680 coupled to high speed interface 640. In other implementations, multiple processors and/or multiple buses may be used, as appropriate, along with multiple memories and types of memory. Also, multiple computing devices 600 may be connected, with each device providing portions of the necessary operations (e.g., as a server bank, a group of blade servers, or a multi-processor system).

The memory 620 stores information non-transitorily within the computing device 600. The memory 620 may be a computer-readable medium, a volatile memory unit(s), or non-volatile memory unit(s). The non-transitory memory 620 may be physical devices used to store programs (e.g., sequences of instructions) or data (e.g., program state information) on a temporary or permanent basis for use by the computing device 600. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 630 is capable of providing mass storage for the computing device 600. In some implementations, the storage device 630 is a computer-readable medium. In various different implementations, the storage device 630 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 620, the storage device 630, or memory on processor 610.

The high speed controller 640 manages bandwidth-intensive operations for the computing device 600, while the low speed controller 660 manages lower bandwidth-intensive operations. Such allocation of duties is exemplary only. In some implementations, the high-speed controller 640 is coupled to the memory 620, the display 680 (e.g., through a graphics processor or accelerator), and to the high-speed expansion ports 650, which may accept various expansion cards (not shown). In some implementations, the low-speed controller 660 is coupled to the storage device 630 and a low-speed expansion port 690. The low-speed expansion port 690, which may include various communication ports (e.g., USB, Bluetooth(R), Ethernet, wireless Ethernet), may be coupled to one or more input/output devices, such as a keyboard, a pointing device, a scanner, or a networking device such as a switch or router, e.g., through a network adapter.

The computing device 600 may be implemented in a number of different forms, as shown in the figure. For example, it may be implemented as a standard server 600a or multiple times in a group of such servers 600a, as a laptop computer 600b, or as part of a rack server system 600c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

In exemplary embodiments of the device and system provided above, in accordance with FIGS. 1-6, the autoinjector 400 delivers one or more therapeutic agents that may be useful in the prevention and/or treatment of nausea and/or vomiting.

As used herein, “treatment” (and variations such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of a condition, alleviation of symptoms, diminishment of any direct or indirect pathological consequences of the condition or treatment, preventing emesis, i.e., by preventing the occurrence of symptoms in whole or in part associated with a condition or side-effects known to accompany a specific treatment, decreasing the rate of progression, amelioration or palliation of the symptoms associated with emesis, such as nausea and/or vomiting, and remission or improved prognosis.

In some embodiments of the disclosure, a therapeutic drug, medicament or agent is used to inhibit or delay development of emesis, i.e. nausea and/or vomiting or to slow the progression of emesis or the symptoms associated with emesis, or to prevent, delay or inhibit the development of emesis, nausea and/or vomiting related to the treatment of a different disease being actively treated.

By “reduce” or “inhibit” is meant the ability to cause an overall decrease of 20%, 30%, 40%, 50%, 60%, 70%, 75%, 80%, 85%, 90%, 95%, or greater. In some embodiments, reduce or inhibit can refer to a relative reduction compared to a reference (e.g., reference level of biological activity (e.g., the number of episodes of nausea and/or vomiting after administration to a subject of a prescribed amount of chemotherapy, for example, a prescribed dose of a chemotherapeutic agent that is known to cause emesis). In some embodiments, reduce or inhibit can refer to the relative reduction of a side effect (i.e. nausea and/or vomiting) associated with a treatment for a condition or disease.

In various embodiments of the present disclosure, a therapeutic drug, medicament or agent known and/or effective for treating or preventing nausea and/or vomiting, or known and/or effective for reducing one or more symptoms associated with nausea and/or vomiting is provided for the treatment and/or prevention of nausea and/or vomiting.

In various embodiments, the device and system for managing nausea and/or vomiting provides for administration of a therapeutically effective amount of a therapeutic drug, medicament or agent known and/or effective for treating or preventing nausea and/or vomiting, or known and/or effective for reducing one or more symptoms associated with nausea and/or vomiting is to the subject.

As used herein, the device and system may administer a therapeutic drug, medicament or agent known and/or effective for treating or preventing nausea and/or vomiting, or known and/or effective for reducing one or more symptoms associated with nausea and/or vomiting to prevent and/or treat vomiting, for example, cyclic vomiting syndrome.

In various embodiments, the device or system used for the prevention and/or treatment of nausea and/or vomiting employs a therapeutic drug, medicament or agent known and/or effective for treating or preventing nausea and/or vomiting, or known and/or effective for reducing one or more symptoms associated with nausea and/or vomiting which have an antiemetic action, and which may inhibit or reduce the number and severity of the occurrence of nausea, and/or vomiting when associated with various stimuli disclosed herein, for example, when a subject has cyclic vomiting syndrome or is administered a chemotherapeutic drug, for example, a chemotherapeutic drug with emetic potential, such as platinum based chemotherapeutics such as cisplatin, oxaliplatin, and carboplatin; irinotecan and other topo isomerase inhibitors used in the treatment of cancer.

In various embodiments, the device or system used for the prevention and/or treatment of nausea and/or vomiting employs a therapeutic drug, medicament or agent known and/or effective for treating or preventing nausea and/or vomiting, or known and/or effective for reducing one or more symptoms associated with nausea and/or vomiting.

The therapeutic drug, medicament or agent known and/or effective for treating or preventing nausea and/or vomiting, or known and/or effective for reducing one or more symptoms associated with nausea and/or vomiting of the present disclosure has preferably low toxicity (e.g., acute toxicity, chronic toxicity, genetic toxicity, reproductive toxicity, cardiac toxicity, carcinogenicity), shows a few side effects, and can be safely administered to a mammal (e.g., human, bovine, horse, dog, cat, monkey, mouse, rat) as an agent for the prophylaxis or treatment of emesis.

“Treatment,” in the context of treating emesis by administering at least one therapeutic drug, medicament or agent known and/or effective for treating or preventing nausea and/or vomiting, or known and/or effective for reducing one or more symptoms associated with nausea and/or vomiting, which may include either or both prophylactic treatment and the treatment of emesis after a subject experiences emesis. Prophylactic treatment includes administration of a therapeutic drug, medicament or agent known and/or effective for treating or preventing nausea and/or vomiting, or known and/or effective for reducing one or more symptoms associated with nausea and/or vomiting using a device and/or system of the present disclosure, before a subject experiences emesis, such as when the subject experiences nausea, as well as administration of the therapeutic drug, medicament or agent known and/or effective for treating or preventing nausea and/or vomiting, or known and/or effective for reducing one or more symptoms associated with nausea and/or vomiting, using a device and/or system of the present disclosure before the subject is exposed to a substance, agent, or event, or before the subject contracts a condition, which results in or is likely to result in the subject experiencing emesis. As used herein, “therapeutically effective amount” refers to an amount of the therapeutic drug, medicament or agent known and/or effective for treating or preventing nausea and/or vomiting, or known and/or effective for reducing one or more symptoms associated with nausea and/or vomiting, sufficient to elicit the desired biological response. In the present disclosure, the desired biological response is treating and/or preventing emesis (nausea and/or vomiting) in a subject in need thereof.

The therapeutic drug, medicament or agent known and/or effective for treating or preventing nausea and/or vomiting, or known and/or effective for reducing one or more symptoms associated with nausea and/or vomiting of the present disclosure is administered using a device and/or system of the present disclosure, which can be used to treat or prevent emesis, for example, when a subject experiences or is about to experience emesis, such as nausea and/or vomiting. In various embodiments, the subject, for example, a mammal, for example, humans, non-human primates, apes, monkeys, laboratory mammals for example, mice, rats, rabbits, guinea-pigs, ferrets, domesticated mammals, such as companion mammals, dogs, cats and horses, and farm mammals, such as cattle, pigs, sheep and goats purely as examples, but not intended to be an exhaustive list, may be monitored and treated with a device and/or system as described herein, the method comprising administering a therapeutic drug, medicament or agent known and/or effective for treating or preventing nausea and/or vomiting, or known and/or effective for reducing one or more symptoms associated with nausea and/or vomiting of the present disclosure. In each of these cases, the methods of the present disclosure are provided to treat or prevent emesis in a subject in need thereof, to reduce or inhibit emesis, to reduce or inhibit a symptom associated with emesis, or to reduce or inhibit a pathological condition or symptom associated with emesis, for example, nausea and/or vomiting.

In order to prevent or treat emesis, the device and/or system of the present disclosure may administer an effective amount of a therapeutic drug, medicament or agent known and/or effective for treating or preventing nausea and/or vomiting, or known and/or effective for reducing one or more symptoms associated with nausea and/or vomiting, in a pharmaceutical composition and administered to a subject/patient (used interchangeably herein) in need thereof. A subject is determined to be in need of treatment with a therapeutic drug, medicament or agent known and/or effective for treating or preventing nausea and/or vomiting, or known and/or effective for reducing one or more symptoms associated with nausea and/or vomiting, by engaging and executing the functions of the device and/or system as described herein. In various embodiments, the system employing the device will be capable of detecting, measuring, and/or quantifying physiological parameters and changes in those parameters that are indicative or predictive of nausea and/or vomiting. The system will be capable of calculating the trend and variability in the measurement data, either through observation of vomiting by the subject, or through a subject's self-reporting of emesis (in the case of a human subject). A patient is determined to be in need of preventative therapy by assessing that the patient is at risk of experiencing emesis due to another medical condition or due to exposure to an agent known to be associated with emesis, such as an infection by a virus or bacteria or chemical agent or radiation as determined using the device and system for preventing such emesis or by treating the emesis experienced or likely to be experienced by the wearer of the device.

The therapeutic drug, medicament or agent known and/or effective for treating or preventing nausea and/or vomiting, or known and/or effective for reducing one or more symptoms associated with nausea and/or vomiting may be administered as an illustrative agent or therapeutic agent using the device and system described herein are beneficial in the therapy of acute, delayed or anticipatory emesis, including emesis induced by chemotherapy, radiation, toxins, viral or bacterial infections, pregnancy, vestibular disorders (e.g. motion sickness, vertigo, dizziness and Meniere's disease), surgery, pain, opioid use and withdrawal, migraine, and variations in intracranial pressure. The uses of this invention are of particular benefit in the therapy of emesis induced by radiation, for example during the treatment of cancer, or radiation sickness, and in the treatment of post-operative nausea and vomiting. Most especially, use of the invention is beneficial in the therapy of emesis induced by antineoplastic (cytotoxic) agents including those routinely used in cancer chemotherapy, emesis induced by other pharmacological agents, for example, alpha-2 adrenoceptor antagonists, such as yohimbine, MK-912 and MK-467, and type IV cyclic nucleotide phosphodiesterase (PDE4) inhibitors, such as RS14203, CT-2450 and rolipram.

Particular examples of chemotherapeutic agents which may cause emesis, nausea and/or vomiting are described, for example, by D. J. Stewart in Nausea and Vomiting: Recent Research and Clinical Advances, ed. J. Kucharczyk et al., CRC Press Inc., Boca Raton, Fla., USA, 1991, pages 177-203, especially page 188. Commonly used chemotherapeutic agents which may cause emesis, nausea and/or vomiting, for example, cyclic vomiting syndrome may include cisplatin, carboplatin, oxaliplatin, cyclophosphamide, dacarbazine (DTIC), dactinomycin, mechlorethamine (nitrogen mustard), streptozocin, cyclophosphamide, carmustine (BCNU), irinotecan, and other topoisomerase inhibitors, lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel and chlorambucil (R. J. Gralle et al. in Cancer Treatment Reports, 1984, 68, 163-172). Emesis due to other chemical agents, such as the toxins soman or sarin, or opioid drug usage and/or withdrawal, e.g. morphine, heroin, oxycodone, and the like can also be prevented and/or treated using the device and system of the present disclosure.

The present therapeutic drug, medicament or agent known and/or effective for treating or preventing nausea and/or vomiting, or known and/or effective for reducing one or more symptoms associated with nausea and/or vomiting is administered to a patient in a quantity sufficient to treat or prevent the symptoms and/or underlying etiology associated with emesis in the patient. In a preferred embodiment, the therapeutic drug, medicament or agent known and/or effective for treating or preventing nausea and/or vomiting, or known and/or effective for reducing one or more symptoms associated with nausea and/or vomiting is administered prior to administration of an agent which is likely to cause emesis, such as one or more of the chemotherapeutic agents described above. The present therapeutic drug, medicament or agent known and/or effective for treating or preventing nausea and/or vomiting, or known and/or effective for reducing one or more symptoms associated with nausea and/or vomiting can also be administered in combination with such agents, either in physical combination or in combined therapy through the administration of the present compounds and agents in succession (in any order). Although the present disclosure is useful in any mammal suffering from emesis, a preferred subject is a human.

In some embodiments, the therapeutic drug, medicament or agent known and/or effective for treating or preventing nausea and/or vomiting, or known and/or effective for reducing one or more symptoms associated with nausea and/or vomiting may be administered using a device and/or system as described herein to prevent and/or treat emesis (for example nausea and/or vomiting) when a subject is concomitantly being treated for diabetes and/or obesity. Several known anti-diabetic medicaments are known for causing emesis, for example, Metformin (Glucophage, Glumetza, others), sulfonylureas, meglitinides, thiazolidinediones, DPP-4 inhibitors, SGLT2 inhibitors, and GLP-1 receptor agonists. In some embodiments, methods for treating emesis in a subject, for example in a subject in need thereof, may include administering an effective amount of a therapeutic drug, medicament or agent known and/or effective for treating or preventing nausea and/or vomiting, or known and/or effective for reducing one or more symptoms associated with nausea and/or vomiting to a subject that does not have type-2 diabetes mellitus or a subject that is not taking a medicament to treat type-2 diabetes mellitus while experiencing emesis.

Nausea is a subjective unpleasant feeling in the back of one's throat and stomach that may lead to vomiting. There are many words that describe nausea including, but not limited to: sick to my stomach, queasy, or upset stomach. Nausea can have other symptoms that happen at the same time, such as increased saliva (spit), dizziness, lightheadedness, trouble swallowing, skin temperature changes, and a fast heart rate. Vomiting is also described as “throwing up.” When one vomits, one's stomach muscles contract (squeeze) and push the contents of one's stomach out through their mouth. One might or might not feel nauseated. Retching is when one tries to vomit without bringing anything up from one's stomach. Other words used to describe retching are gagging or dry heaves. Nausea and vomiting often happen at the same time, but they can be 2 different conditions that may be mutually exclusive or mutually associated. Some chemotherapy drugs are more likely to cause nausea and vomiting than others. Doctors classify chemotherapy drugs according to their emetogenic potential (how likely the drug will cause nausea or vomiting) as high, moderate, low, or minimal risk.

The therapeutic drug, medicament or agent known and/or effective for treating or preventing nausea and/or vomiting, or known and/or effective for reducing one or more symptoms associated with nausea and/or vomiting of the present disclosure may be used as a preventive/therapeutic agent for vomiting and/or nausea caused, for example, by clinical pathological conditions or causes described in the following (1) to (10). Additionally, the therapeutic drug, medicament or agent known and/or effective for treating or preventing nausea and/or vomiting, or known and/or effective for reducing one or more symptoms associated with nausea and/or vomiting of the present disclosure may be used as a preventive/therapeutic agent for chronic unexplained nausea and vomiting. The vomiting or nausea also includes imminent unpleasant sensations of wanting to eject the contents of the stomach through the mouth such as feeling queasy and retching, and may also be accompanied by autonomic symptoms such as facial pallor, cold sweat, salivary secretion, tachycardia, and diarrhea. The vomiting also includes: cyclic vomiting syndrome, acute vomiting, protracted vomiting, and anticipatory vomiting.
(1) Diseases accompanied by vomiting or nausea such as gastroparesis, gastrointestinal hypomotility, peritonitis, abdominal tumor, constipation, gastrointestinal obstruction, chronic intestinal pseudo-obstruction, functional dyspepsia, cyclic vomiting syndrome, chronic unexplained nausea and vomiting, acute pancreatitis, chronic pancreatitis, hepatitis, hyperkalemia, cerebral edema, intracranial lesion, metabolic disorder, gastritis caused by an infection, postoperative disease, myocardial infarction, migraine, intracranial hypertension, and intracranial hypotension (e.g., altitude sickness);
(2) Vomiting and/or nausea induced by chemotherapeutic drugs such as (i) alkylating agents (e.g., cyclophosphamide, carmustine, lomustine, chlorambucil, streptozocin, dacarbazine, ifosfamide, temozolomide, busulfan, bendamustine, and melphalan), cytotoxic antibiotics (e.g., dactinomycin, doxorubicin, mitomycin-C, bleomycin, epirubicin, actinomycin D, amrubicin, idarubicin, daunorubicin, and pirarubicin), antimetabolic agents (e.g., cytarabine, methotrexate, 5-fluorouracil, enocitabine, and clofarabine), vinca alkaloids (e.g., etoposide, vinblastine, and vincristine), other chemotherapeutic agents such as cisplatin, procarbazine, hydroxyurea, azacytidine, irinotecan, interferon α, interleukin-2, oxaliplatin, carboplatin, nedaplatin, and miriplatin; (ii) opioid analgesics (e.g., morphine); (iii) dopamine receptor D1D2 agonists (e.g., apomorphine); (iv) cannabis and cannabinoid products including cannabis hyperemesis syndrome;
(3) Vomiting or nausea caused by radiation sickness or radiation therapy for the chest, the abdomen, or the like used to treat cancers;
(4) Vomiting or nausea caused by a poisonous substance or a toxin;
(5) Vomiting and nausea caused by pregnancy including hyperemesis gravidarium;
(6) Vomiting and nausea caused by a vestibular disorder such as motion sickness or dizziness;
(7) Opioid withdrawal;
(8) Pregnancy including hyperemesis gravidarium;
(9) A vestibular disorder such as motion sickness or dizziness; and
(10) A physical injury causing local, systemic, acute or chronic pain.

These causes of emesis, or nausea, or vomiting are not meant to be exhaustive. Other conditions, activities, side effects may cause emesis, for example, nausea and/or vomiting. Nausea can be measured in ways known to the art, such as through the use of a visual analog scale (VAS).

The therapeutic drug, medicament or agent known and/or effective for treating or preventing nausea and/or vomiting, or known and/or effective for reducing one or more symptoms associated with nausea and/or vomiting of the present disclosure can also be used for secondary prevention or suppression of progression of the abovementioned various diseases (e.g., cardiovascular events such as myocardial infarction and the like). In addition, the device comprising a therapeutic drug, medicament or agent known and/or effective for treating or preventing nausea and/or vomiting, or known and/or effective for reducing one or more symptoms associated with nausea and/or vomiting of the present disclosure is also useful as a feeding suppressant and a weight reducing agent. The therapeutic drug, medicament or agent known and/or effective for treating or preventing nausea and/or vomiting, or known and/or effective for reducing one or more symptoms associated with nausea and/or vomiting of the present disclosure can also be used in combination with a diet therapy (e.g., diet therapy for diabetes), and an exercise therapy.

### Example

Study Design: This was an observational, electronic data capture (EDC) study to determine the predictive value of electrodermalactivity (EDA), heart rate (HR), actigraphy, skin electrical properties, or skin/surface temperature, as captured by 2 wearable devices, in predicting the onset of vomiting in adult subjects with Cyclic Vomiting Syndrome (CVS). The study enrolled 50 (maximum of 100) adult subjects with CVS, as defined by Rome IV criteria (at least 2 acute-onset vomiting episodes in the past 6 months, each occurring at least 1 week apart, and persisting for less than 1 week) and was about 6 months in duration, inclusive of screening. There was no treatment administered in this study. Subjects had the option of completing both the screening visit (Visit 1) and the end of study visit (Visit 2) in-person at the study site or completing one or both visits remotely. Subjects who elected to complete the screening visit remotely received their wearable electronic devices (Apple Watch and Empatica Embrace) and iPhonesin in the mail. Subjects who elected to complete the end of study visit remotely were issued a prepaid mailer for return of all electronic devices. During the screening visit, subjects were informed of the study procedures and obligations and provided with an informed consent form for review and signature; subjects consenting remotely electronically signed their consent forms. Following assessment of eligibility criteria, concomitant medications and medical conditions were recorded, and a medical history was collected. Eligible subjects were required to confirm that they were the only ones wearing the devices (Apple Watch and Empatica Embrace).

Demographics, height, and weight assessments were also taken during the screening visit. A handedness test (4-Item Edinburgh Handedness Inventory; [1]) was performed on each subject to identify the dominant and nondominant wrist for placement, respectively, of the Empatica Embrace and Apple Watch. In addition to the 2 wearable devices, subjects also received iPhones on which they were asked to complete electronic patient-reported outcomes (ePRO) either daily (symptoms, school/work absenteeism due to CVS, changes in acute CVS therapies, healthcare resource utilization [HRU], and cannabis use) or weekly (impact on health-related quality of life using the Patient-Reported Outcome Measurement Information System-29 [PROMIS-29]). Subjects were asked on a weekly basis, in the ePRO, whether they experienced any local skin reactions (eg, rash, skin irritation) that occured at the site of the wearable devices. Subjects were trained on the use of the wearable devices and the iPhones during their screening visit.

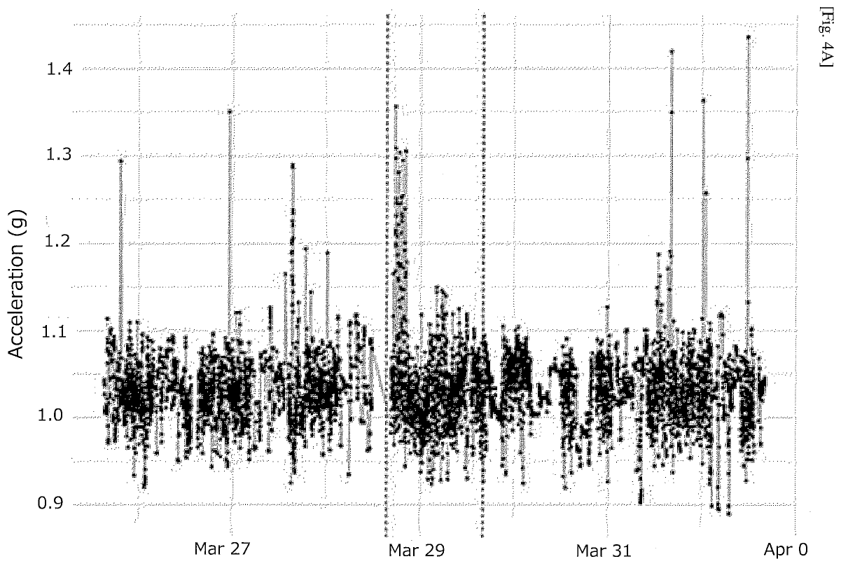

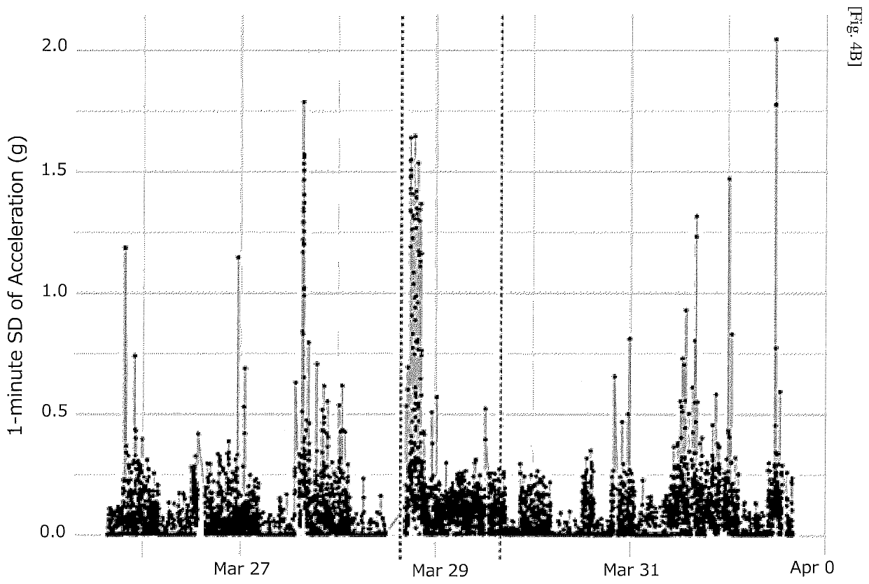

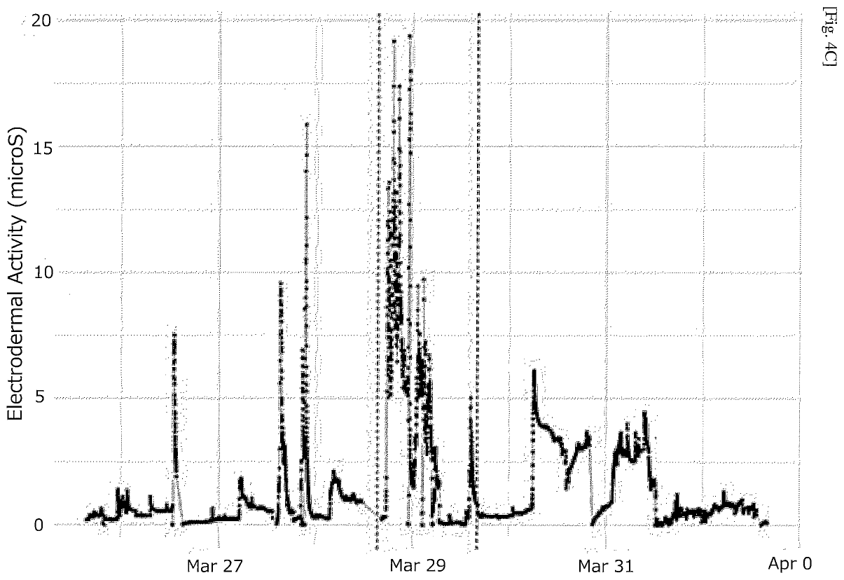

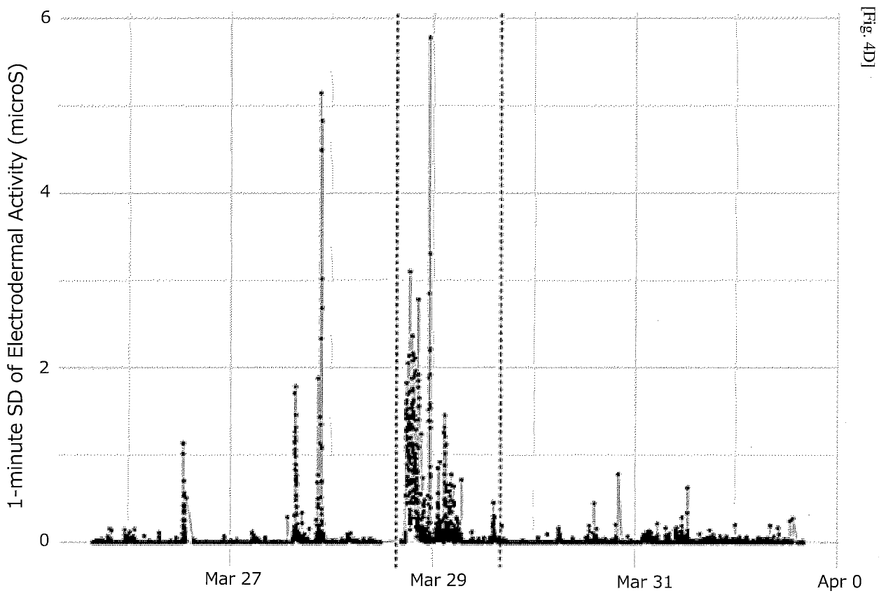

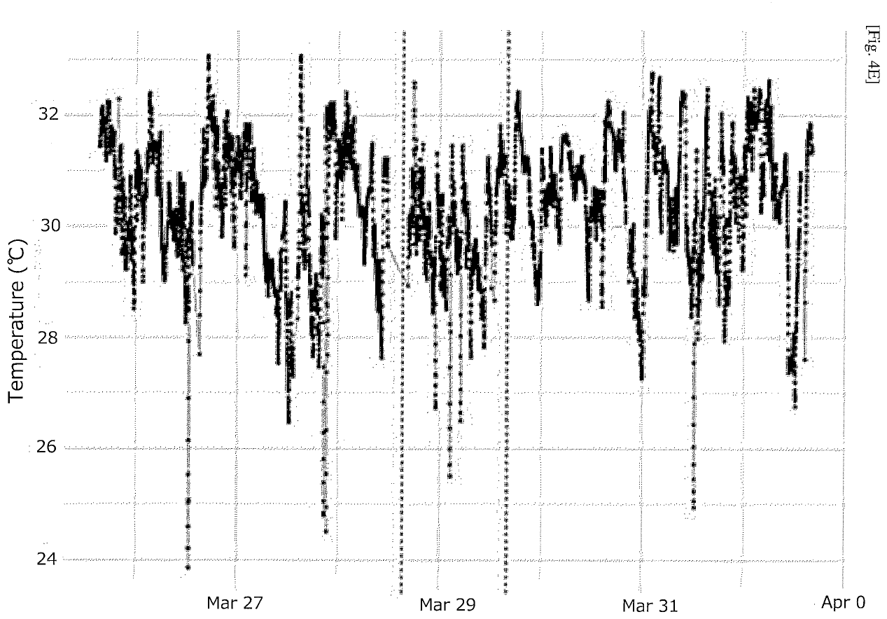

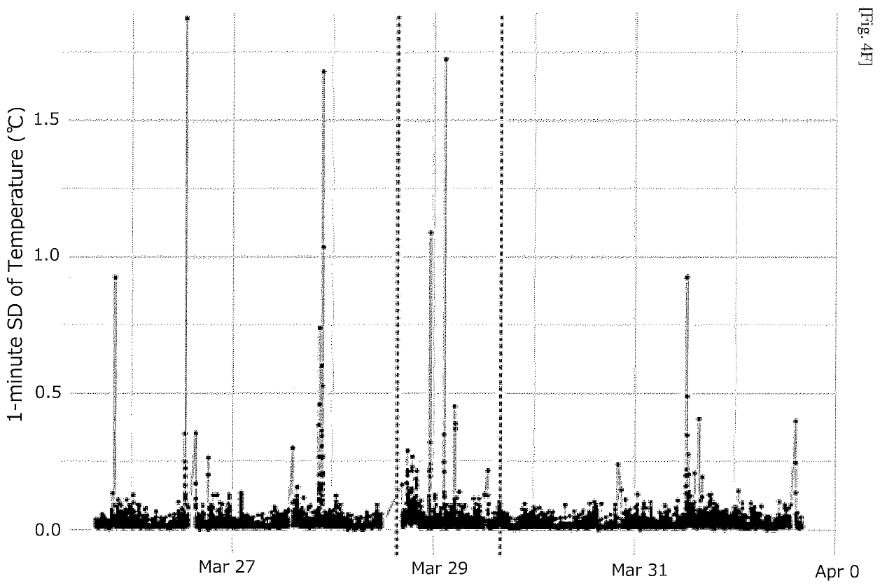

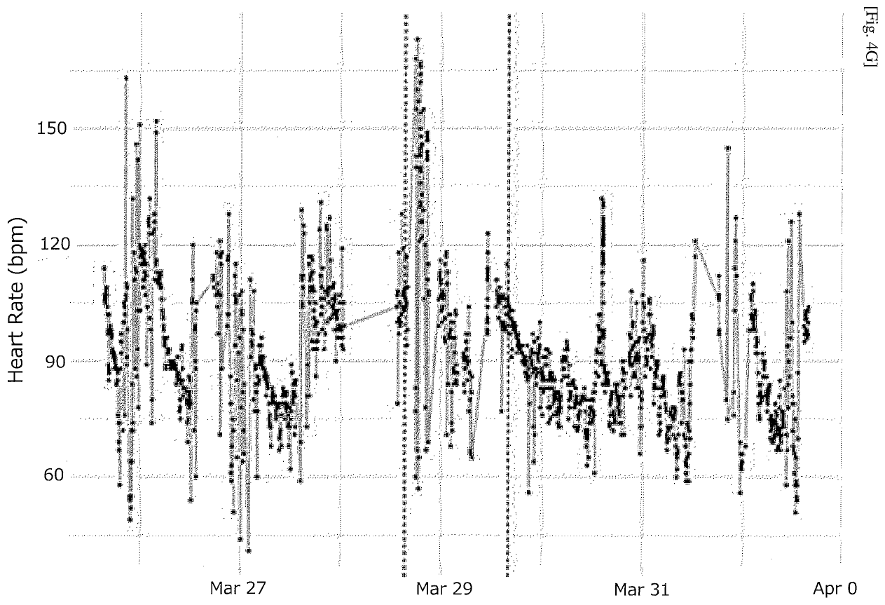

FIGS. 4A-4G show one week actigraphy activity data, electrodermal activity data, heart rate data, and skin temperature collected from a patient participated in the study of the Example. In between the dashed line in each FIG. is a day where the patient vomited 25 times and retched 19 times.

From the FIGS., we can see the there is a difference before the vomiting period and after the vomiting period on each biomarker, especially on electrodermal activity and actigraphy.

While this specification contains many specifics, these should not be construed as limitations on the scope of the disclosure or of what may be claimed, but rather as descriptions of features specific to particular implementations of the disclosure. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multi-tasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

A number of implementations have been described. Nevertheless, it will be understood that various modifications may be made without departing from the spirit and scope of the disclosure. Accordingly, other implementations are within the scope of the following claims. For example, the actions recited in the claims can be performed in a different order and still achieve desirable results.

1. A computer-implemented method (500) when executed by data processing hardware of a computing device (300) causes the data processing hardware to perform operations comprising:
receiving (502), from one or more monitoring devices (200), patient parameter data (212) of a patient (10), the patient parameter data (212) collected by the one or more monitoring devices (200);
determining, (504), based on the patient parameter data (212) of the patient (10), whether the patient (10) is experiencing or about to experience an episode of nausea; and
when the patient is experiencing or about to experience an episode of nausea.

2. The method of claim 1, wherein:
when the patient is experiencing or about to experience an episode of nausea, the operation further comprises communicating (506) one or more therapy signals (302, 304, 306, 308) to a receiver device (12, 21, 400) in communication with the computing device, the receiver device (12, 21, 400) configured to contribute to providing nausea therapy to the patient (10).

3. The method (500) of claim 1, wherein:
the one or more therapy signals (302, 304, 306, 308) include a nausea therapy delivering device activation signal (302);
the receiver device (12, 21, 400) comprises a nausea therapy delivery device (400) associated with the patient (10); and
communicating the one or more therapy signals (302, 304, 306, 308) comprises communicating the nausea therapy delivering device activation signal (302) to the nausea therapy delivery device (400).

4. The method (500) of claim 3, wherein the nausea therapy delivering device activation signal (302) when received by the nausea therapy delivery device (400) is configured to activate the nausea therapy delivery device (400) and cause the nausea therapy delivery device (400) to deliver a nausea therapeutic to the patient (10).

5. The method (500) of claim 3, wherein the nausea therapy delivering device (400) comprises: an automatic injector; an infusion pump; an inhaler; a transcutaneous patch; a sub-dermal implantable pump; or a small molecule, biologic, cell therapy administrator.

6. The method (500) of claim 1, wherein:
the one or more therapy signals (302, 304, 306, 308) comprise a notification signal (304);
the receiver device (12, 21, 400) comprises a user computing device (12); and
communicating the one or more therapy signals (302, 304, 306, 308) comprises communicating the notification signal (304) to the user computing device (12).

7. The method (500) of claim 6, wherein the notification signal (304) when received by the user computing device (12) is configured to display a message on a user interface (240), the message prompting the patient (10) to self-administer a nausea therapeutic via a nausea therapy delivery device (400).

8. The method (500) of claim 7, wherein the operations further comprise, after communicating the notification signal (304) to the user computing device (12), receiving a confirmation signal from the user computing device (12) indicating that the patient (10) self-administered the nausea therapeutic via the nausea therapy delivery device (400).

9. The method (500) of claim 1, wherein the patient parameter data (212) comprises one or more physiologic parameters of the patient (10).

10. The method (500) of claim 9, wherein one or more physiologic parameters of the patient (10) comprise at least one of: electrodermal activity, skin temperature, core temperature, electrocardiography, blood pressure, heart rate, heartrate variability, voice or speech patterns, or actigraphy.

11. The method (500) of claim 1, wherein the patient parameter data (212) comprises at least one of demographic parameters or anthropometric parameters.

12. The method (500) of claim 11, wherein the at least one of demographic parameters or anthropometric parameters comprises at least one of: age, gender, height, weight, body mass index, or disease state.

13. The method (500) of claim 1, wherein the patient parameter data (212) comprises at least one of: electrodermal activity, skin temperature, core temperature, electrocardiography, blood pressure, heart rate, heartrate variability, actigraphy, voice or speech patterns, age, gender, height, weight, body mass index, disease state, or patient reported outcome (PRO).

14. The method (500) of claim 1, wherein determining, quantitively assessing, or qualitatively assessing whether the patient (10) is experiencing or about to experience an episode of nausea comprises executing a predictive model (320) configured to receive the patient parameter data and generate an indicator score (330) indicating whether the patient (10) is experiencing or about to experience the episode of nausea.

15. The method (500) of claim 14, wherein the predictive model (320) is trained on a corpus of training parameters (212_{T}) associated with training parameter data collected from a first group of subjects who suffer from episodes of nausea and a second group of subjects who do not suffer from episodes of nausea.

16. A management system (100) comprising:
data processing hardware of a computing device (300); and
memory hardware of the computing device (300) in communication with the data processing hardware, the memory hardware storing instructions that when executed on the data processing hardware cause the data processing hardware to perform operations comprising:
receiving (502), from one or more monitoring devices (200), patient parameter data (212) of a patient (10), the patient parameter data (212) collected by the one or more monitoring devices (200); and
determining (504), based on the patient parameter data (212) of the patient (10), whether the patient (10) is experiencing or about to experience an episode of nausea.

17. The management system (100) of claim 16, wherein, when the patient is experiencing or about to experience an episode of nausea, the operations further comprise communicating (506) one or more therapy signals (302, 304, 306, 308) to a receiver device (12, 21, 400) in communication with the computing device, the receiver device (12, 21, 400) configured to contribute to providing nausea therapy to the patient (10).

18. The management system (100) of claim 17, wherein:
the one or more therapy signals (302, 304, 306, 308) include a nausea therapy delivering device activation signal (302);
the receiver device (12, 21, 400) comprises a nausea therapy delivery device (400) associated with the patient (10); and
communicating the one or more therapy signals (302, 304, 306, 308) comprises communicating the nausea therapy delivering device activation signal (302) to the nausea therapy delivery device (400).

19. The management system (100) of claim 18, wherein the nausea therapy delivering device activation signal (302) when received by the nausea therapy delivery device (400) is configured to activate the nausea therapy delivery device (400) and cause the nausea therapy delivery device (400) to deliver a nausea therapeutic to the patient (10).

20. The management system (100) of claim 18, wherein the nausea therapy delivering device (400) comprises: an automatic injector; an infusion pump; an inhaler; a transcutaneous patch; a sub-dermal implantable pump; or a small molecule, biologic, cell therapy administrator.

21. The management system (100) of claim 17, wherein:
the one or more therapy signals (302, 304, 306, 308) comprise a notification signal (304);
the receiver device (12, 21, 400) comprises a user computing device (12); and
communicating the one or more therapy signals (302, 304, 306, 308) comprises communicating the notification signal (304) to the user computing device (12).

22. The management system (100) of claim 21, wherein the notification signal (304) when received by the user computing device (12) is configured to display a message on a user interface (240), the message prompting the patient (10) to self-administer a nausea therapeutic via a nausea therapy delivery device (400).

23. The management system (100) of claim 22, wherein the operations further comprise, after communicating the notification signal (304) to the user computing device (12), receiving a confirmation signal from the user computing device (12) indicating that the patient (10) self-administered the nausea therapeutic via the nausea therapy delivery device (400).

24. The management system (100) of claim 16, wherein the patient parameter data (212) comprises one or more physiologic parameters of the patient (10).

25. The management system (100) of claim 24, wherein one or more physiologic parameters of the patient (10) comprise at least one of: electrodermal activity, skin temperature, core temperature, electrocardiography, blood pressure, heart rate, heartrate variability, voice or speech patterns, or actigraphy.

26. The management system (100) of claim 16, wherein the patient parameter data (212) comprises at least one of demographic parameters or anthropometric parameters.

27. The management system (100) of claim 26, wherein the at least one of demographic parameters or anthropometric parameters comprises at least one of: age, gender, height, weight, body mass index, or disease state.

28. The management system (100) of claim 16, wherein determining whether the patient (10) is experiencing or about to experience an episode of nausea comprises executing a predictive model (320) configured to receive the patient parameter data and generate an indicator score (330) indicating whether the patient (10) is experiencing or about to experience the episode of nausea.

29. The management system (100) of claim 28, wherein the predictive model (320) is trained on a corpus of training parameters (212_{T}) associated with training parameter data collected from a first group of subjects who suffer from episodes of nausea and a second group of subjects who do not suffer from episodes of nausea.

30. A method for treating a disease or preventing a disease progression in a subject comprising:
(a) a monitor device for tracking one or more symptoms, clinical manifestations, or therapy signals of the disease;
(b) a computing device for analyzing the symptoms, clinical manifestations, or therapy signals of the disease and to determining, quantitively assessing, or qualitatively assessing whether the subject is experiencing or about to experience an episode of the disease;
(c) a receiver device in communication with the computing device, and optionally;
(d) a therapy delivery device for enabling administration of a therapeutic agent to the subject in need thereof for treating or preventing the disease; wherein the disease is nausea, retch, and/or vomiting.

31. The method of claim 30, wherein the method monitors the patient's status either continuously, on-demand, prodromal, or the onset of symptoms of nausea and/or vomiting.

32. The method of claim 30, wherein the method comprising monitoring, computing, assessing, or receiving input of one or more physiologic, demographic, and/or anthropometric characteristics.

33. The method of claim 32, wherein the physiologic, demographic, and/or anthropometric characteristics comprises: electrodermal activity, temperature, electrocardiography, blood pressure, heart rate, heartrate variability, actigraphy, voice or speech patterns, age, gender, height, weight, body mass index, sleep activity, diagnosis of inflammatory bowel disease, ulcerative colitis, or cyclic vomiting syndrome.

34. The method of claim 30, wherein the method further comprising identifying, counting, or quantitatively assessing the number of emetic events.

35. The method of claim 34, wherein the emetic events is nausea, retch, or vomiting.

36. The method of claim 30, wherein the delivery device comprises at least one of: an automatic injector, an infusion pump, an inhaler, a transcutaneous patch, a sub-dermal implantable pump, or a small molecule, biologic, cell therapy administrator.

37. The method of 30, wherein method comprises a computer-implemented method when executed by data processing hardware of a computing device causes the data processing hardware to perform operations including:
(1) receiving patient parameter data of a patient collected by the one or more monitoring devices;
(2) determining, quantitively assessing, or qualitatively assessing, based on the patient parameter data of the patient, whether the patient is experiencing or about to experience an episode of nausea and/or vomiting; and optionally;
(3) when the patient is experiencing or about to experience an episode of nausea or vomiting, communicating one or more therapy signals to a receiver device in communication with the computing device, the receiver device configured to contribute to providing nausea therapy to the patient.

38. The method of claim 37, wherein the method includes a therapy delivering device activation signal.

39. The method of claim 37, wherein the receiver device includes a therapy delivery device associated with the patient.

40. The method of claim 37, wherein the operation of communicating the symptoms, clinical manifestations, or therapy signals includes communicating the therapy delivering device activation signal to the therapy delivery device.

41. The method of claim 38, wherein the therapy delivering device activation signal when received by the therapy delivery device is configured to activate the therapy delivery device and cause the therapy delivery device to deliver a therapeutic agent to the patient.

42. The method of claim 41, the therapy agent is an agent for treating or preventing nausea or vomiting.

43. The method of claim 42, wherein the method further comprises, after communicating the therapy delivering device activation signal to the therapy delivery device, receiving a confirmation single reporting that the therapy delivery device delivered the therapeutic agent to the patient.

44. The method of claim 37, the therapy delivery device includes an automatic injector; an infusion pump; an inhaler; a transcutaneous patch; a sub-dermal implantable pump; or a small molecule, biologic, cell therapy administrator.

45. The method of claim 37, wherein the therapy signals include a notification signal; wherein the receiver device includes a user computing device, wherein the operation of communicating the therapy signals includes communicating a notification signal to a user computing device.

46. The method of claim 45, wherein the notification signal when received by the user computing device is configured to display a message on a user interface, wherein the message prompts the patient to self-administer a therapeutic agent via a therapy delivery device.

47. The method of claim 46, wherein the therapy agent is an agent for treating or preventing nausea or vomiting.

48. The method of claim 46, further includes, after communicating the notification signal to the user computing device, receiving a confirmation signal from the user computing device indicating that the patient self-administered the therapeutic agent via the nausea therapy delivery device.

49. The method of claim 37, wherein the therapy signals include a report signal, wherein the receiver device includes a healthcare provider computing device associated with a health care provider treating the patient, wherein the operation of communicating the therapy signals includes communicating a report signal to a health care provider computing device.

50. The method of claim 49, wherein the report signal indicates that the patient is experiencing or about to experience the episode of nausea.

51. The method of claim 50, wherein the therapy signals includes a recommendation signal, wherein the recommendation signal when received by the health care provider computing device causes the health care provider computing device to instruct the health care provider to administer a therapeutic agent to the patient.

52. The method of claim 51, wherein the therapy agent is an agent for treating or preventing nausea or vomiting.

53. The method claim 37, wherein the patient parameter data includes one or more parameters of the patient comprising electrodermal activity, skin temperature, core temperature, electrocardiography, blood pressure, heart rate, voice or speech patterns, or heartrate variability.

54. The method claim 37, wherein the patient parameter data includes one or more parameters of the patient comprising age, gender, height, weight, body mass index, or disease state.

55. The method of claim 37, wherein the operation of determining whether the patient is experiencing or about to experience an episode of nausea or vomiting includes executing a predictive model configured to receive the patient parameter data and generate an indicator score indicating whether the patient is experiencing or about to experience the episode.

56. The method of claim 55, wherein the predictive model is trained on a corpus of training parameters associated with training parameter data collected from a first group of subjects who suffer from episodes of nausea or vomiting and a second group of subjects who do not suffer from episodes of nausea or vomiting.

57. The method of claim 30, wherein the method provides a management system including data processing hardware of a computing device and memory hardware of the computing device in communication with the data processing hardware.

58. The method of claim 57, wherein the memory hardware stores instructions that when executed on the data processing hardware causes the data processing hardware to perform operations including: receiving, from one or more monitoring devices, patient parameter data of a patient, the patient parameter data collected by the one or more monitoring devices; determining, based on the patient parameter data of the patient, whether the patient is experiencing or about to experience an episode of nausea; and when the patient is experiencing or about to experience an episode of nausea, communicating one or more therapy signals to a receiver device in communication with the computing device, the receiver device configured to contribute to providing nausea therapy to the patient.

59. The method of claim 30, the method includes one or more of the following optional features:
(1) the one or more therapy signals include a therapy delivering device activation signal;
(2) the receiver device includes a therapy delivery device associated with the patient who is experiencing or about to experience an episode of nausea and/or vomiting;
(3) the operation of communicating the therapy signals includes communicating the therapy delivery device activation signal to the therapy delivery device; and optionally
(4) the therapy delivery device activation signal when received by the therapy delivery device is configured to activate the therapy delivery device and cause the therapy delivery device (400) to deliver a therapeutic agent to the patient;
wherein the therapy delivery device includes an automatic injector, an infusion pump, an inhaler, a transcutaneous patch, a sub-dermal implantable pump, or a small molecule, biologic, cell therapy administrator.

60. The method of claim 59, wherein the method further includes (5) receiving a confirmation single reporting that the therapy delivering device delivered the therapeutic agent to the patient.
